(19) United States
(12) Patent Application Publication
(10) Pub. No.: US 2025/0127886 A1
(43) Pub. Date: Apr. 24, 2025

(54) ASSESSMENT AND TREATMENT OF CHRONIC HEPATITIS B

(71) Applicant: BRII BIOSCIENCES, INC., Durham, NC (US)

(72) Inventors: Zhi Hong, Durham, NC (US); Yun Ji, Durham, NC (US); Ying Tan, Durham, NC (US)

(21) Appl. No.: 18/988,667

(22) Filed: Dec. 19, 2024

Related U.S. Application Data
(63) Continuation of application No. PCT/US2024/035025, filed on Jun. 21, 2024.
(60) Provisional application No. 63/522,851, filed on Jun. 23, 2023.

(51) Int. Cl. A61K 39/29; A61K 39/00; A61P 31/20; C12N 7/00; C12N 15/113; G01N 33/68

(52) U.S. Cl. CPC A61K 39/292; A61P 31/20; C12N 7/00; C12N 15/1131; G01N 33/6854; A61K 2039/5258; A61K 2039/545; A61K 2039/572; C12N 2310/14; C12N 2710/00023; C12N 2710/00034; G01N 2333/02; G01N 2800/52

(57) ABSTRACT

Provided herein are methods for administering a hepatitis B virus surface antigen (HBsAg) to subjects with chronic hepatitis B to distinguish between responder subjects who respond to the HBsAg administration with an increase in an HBV antibody levels and non-responder subjects, and treating chronic hepatitis B in the responder subjects with an HBV therapy. Methods of excluding non-responder subjects from a clinical study are also provided.

### CROSS REFERENCE TO RELATED APPLICATIONS

This application claims the benefit under 35 U.S.C. § 119 (e) of the U.S. Provisional Application Ser. No. 63/522,851, filed Jun. 23, 2023, the content of which is hereby incorporated by reference in its entirety.

### FIELD

Provided herein are methods for treating chronic hepatitis B which comprise the administration of a hepatitis B surface antigen prior to administration of a second therapy.

### BACKGROUND

Chronic hepatitis B (CHB) arising from a long-term infection of the hepatitis B virus (HBV) remains a public health problem affecting an estimated 296 million people worldwide and represents a major contributing factor to development of cirrhosis and its complications including hepatocellular carcinoma and end-stage liver disease. People with CHB often show weak or absent virus specific antibody response or T-cell reactivity, which is described as the “exhaustion” state characterized by poor effector antibody response or cytotoxic activity, impaired cytokine production and sustained expression of multiple inhibitory receptors, which impedes the clearance of virus and recovery from hepatitis (Ye et al., 2015, Cell Death & Disease, 6(3):e1694).

Current therapies for CHB suppress viral HBV DNA, but rarely eradicate the virus. These therapies include (i) nucleoside/nucleotide reverse transcriptase inhibitors (NrtIs), such as lamivudine, entecavir, tenofovir alafenamide, tenofovir disoproxil fumarate, adefovir dipivoxil, and telbivudine; and (ii) the immunomodulatory agent pegylated interferon alfa (PEG-IFNα). Prolonged treatment with NrtI therapy has led to the emergence of HBV strains that confer resistance to NrtIs. Moreover, viral HBV cccDNA is not cleared from the infected cell with NrtI therapy, and viral protein production, in particular the production of HBV surface antigen (HBsAg), may continue as measured in blood (Scaglione and Lok, 2012, Gastroenterology 142:1360-1368). PEG-IFNα therapy has a limited treatment course and the responders to PEG-IFNα may maintain a virologic or serologic response, e.g., clearance of HBsAg in serum, after drug withdrawal, unfortunately, there is relatively low response rate to PEG-IFNα therapy.

The failure of NrtI therapy to produce a functional cure and the limitations of PEG-IFNα therapy highlight the need for effective, well-tolerated HBV therapies.

### SUMMARY

In accordance with one embodiment of the present disclosure, provided is a method for treating a hepatitis B viral (HBV) infection in a subject with chronic hepatitis B, the method comprising a) administering to the subject an HBV surface antigen (HBsAg); and b) at least 4 weeks after an initial dose of the HBsAg, administering to the subject an HBV therapy that is not the HBsAg.

In some embodiments, an initial dose of the HBV therapy is administered after at least 2 doses of the HBsAg, or after at least 3, 4, or 5 doses of the HBsAg. In some embodiments, the initial dose of the HBV therapy is administered at least 8 weeks after the initial dose of the HBsAg, or at least 9, 10, 11, 12, 13, 14, or 15 weeks after the initial dose of the HBsAg.

In some embodiments, the subject, prior to administration of the HBsAg, has not been treated with the HBV therapy. In some embodiments, the subject, prior to administration of the HBsAg, has not been treated with an interferon. In some embodiments, the subject, prior to administration of the HBsAg, has not been treated for the HBV infection.

In some embodiments, the HBsAg is administered at 20 μg to 100 μg per dose once every two weeks (Q2W), once every three weeks (Q3W) or once every four weeks (Q4W).

In some embodiments, the HBV therapy is selected from the group consisting of an anti-HBsAg siRNA, interferon alfa (IFNα), pegylated interferon alfa (PEG-IFNα), an HBV-neutralizing mAb, and combinations thereof.

In some embodiments, the HBV therapy comprises an anti-HBsAg siRNA. In some embodiments, the anti-HBsAg siRNA is administered at 100 mg to 400 mg per dose once every two weeks (Q2W), once every three weeks (Q3W), once every four weeks (Q4W), once every five weeks (Q5W) or once every six weeks (Q6W).

In some embodiments, the HBV therapy further comprises a pegylated interferon alfa (PEG-IFNα). In some embodiments, the PEG-IFNα is administered at 50 to 500 μg once every week, 100 to 300 μg once every week or 180 μg once every week.

Also provided, in one embodiment, is a method for treating a hepatitis B viral (HBV) infection in a subject with chronic hepatitis B, the method comprising administering to the subject 20 μg to 100 μg per dose of an HBV surface antigen (HBsAg) once every two weeks (Q2W), once every three weeks (Q3W) or once every four weeks (Q4W) and 100 mg to 400 mg per dose of an anti-HBsAg siRNA once every two weeks (Q2W), once every three weeks (Q3W), once every four weeks (Q4W), once every five weeks (Q5W) or once every six weeks (Q6W), wherein the treatment comprises at least four (or five, six, seven, eight or ten) doses for each of the HBsAg and the siRNA.

In some embodiments, the HBsAg comprises virus-like particles (VLPs) comprising HBV surface envelope protein Pre-S1, Pre-S2 or S. In some embodiments, the VLPs comprises HBV surface envelope proteins Pre-S1, Pre-S2 and S.

In some embodiments, the siRNA is elebsiran.

In some embodiments, the subject is further administered a nucleoside/nucleotide reverse transcriptase inhibitor (NrtI). In some embodiments, the method further comprises discontinuing the administration of the NrtI if the subject has met one or more of discontinuation criteria. In some embodiments, the discontinuation criteria comprise (a) HBsAg<LLOQ (lower limit of quantification), (b) HBV DNA<LLOQ, (c) undetectable HBcAg, and (d) ALT (alanine aminotransferase)≤2× ULN (upper limit of normal).

In one aspect, provided herein is a method for excluding subjects with chronic hepatitis B (CHB) from a clinical study comprising (a) screening a group of CHB subjects by administering an HBV surface antigen (HBsAg) to the CHB subjects, wherein each CHB subject has a predetermined baseline HBV antibody concentration prior to administration of the HBsAg and wherein each CHB subject who does not respond to the HBsAg administration with a post-baseline HBV antibody concentration greater than their baseline HBV antibody concentration is excluded from the clinical study, and (b) conducting the clinical study wherein a HBV therapy is administered to CHB subjects who are not excluded from the clinical study.

In some embodiments, each CHB subject who does not respond to the HBsAg administration with a post-baseline HBV antibody concentration 5 times greater than their baseline HBV antibody concentration is excluded from the clinical study.

In some embodiments, (i) each CHB subject who has a predetermined baseline HBV antibody concentration at or below a lower limit of detection (e.g., ≤2 IU/L), the CHB subject is excluded from the clinical trial unless the CHB subject's post-baseline HBV antibody concentration above the lower limit of detection (e.g., >2 IU/L), and (ii) each CHB subject who has a predetermined baseline HBV antibody concentration above the lower limit of detection (e.g., >2 IU/L), the CHB subject is excluded from the clinical trial unless the subject's post-baseline HBV antibody concentration is at least 2 times greater than their predetermined baseline HBV antibody concentration.

In some embodiments, a CHB subject is excluded from the clinical trial unless the CHB subject has a post-baseline HBV antibody concentration of greater than 4 IU/L, greater than 5 IU/L, greater than 10 IU/L, greater than 12 IU/L, greater than 15 IU/L, greater than 20 IU/L, greater than 25 IU/L, greater than 30 IU/L, greater than 50 IU/L, greater than 100 IU/L, greater than 150 IU/L, greater than 200 IU/L, greater than 250 IU/L, or greater than 300 IU/L.

In some embodiments, the CHB subjects are virally suppressed with a nucleoside/nucleotide reverse transcriptase inhibitor (NrtI) prior to administration of the HBsAg.

In some embodiments, the HBV antibody concentration is a serum anti-HBS concentration.

In some embodiments, the HBV therapy excludes the HBsAg.

In one aspect, provided herein is a method for selecting a subject with chronic hepatitis B (CHB) for a hepatitis B virus (HBV) therapy comprising: (a) determining a baseline HBV antibody concentration in a subject with CHB prior to administering an HBV surface antigen (HBsAg) to the subject; (b) administering the HBsAg to the subject; (c) determining a post-baseline HBV antibody concentration in the subject after being administered the HBsAg, wherein the subject is selected for an HBV therapy if the post-baseline HBV antibody concentration is greater than the baseline HBV antibody concentration, otherwise the subject is excluded from the HBV therapy; and (d) administering the HBV therapy to the selected subject.

In some embodiments, the subject is selected for an HBV therapy if the post-baseline HBV antibody concentration is 5 times greater than the baseline HBV antibody concentration, otherwise the subject is excluded from the HBV therapy.

In some embodiments, the subject is selected for the HBV therapy if (i) the subject's baseline HBV antibody concentration is at or below a lower limit of detection (e.g., ≤2 IU/L), and the subject's post-baseline HBV antibody concentration is above the lower limit of detection (e.g., >2 IU/L), or (ii) the subject's baseline HBV antibody concentration is above the lower limit of detection (e.g., >2 IU/L), and the subject's post-baseline HBV antibody concentration is at least 2 times greater than the subject's baseline HBV antibody concentration, otherwise the subject is excluded from the HBV therapy.

In some embodiments, the subject is virally suppressed with a nucleoside/nucleotide reverse transcriptase inhibitor (NrtI) prior to administration of the HBsAg.

In some embodiments, the baseline and post-baseline HBV antibody concentrations are anti-HBs concentrations determined from serum samples collected from the subject. In some embodiments, the HBV therapy excludes the HBsAg. In some embodiments, the HBV therapy is an siRNA, interferon alfa (IFNα), pegylated interferon alfa (PEG-IFNα), an HBV-neutralizing mAb, or a combination thereof.

In one aspect, provided herein is a method for treating an HBV infection in a subject with chronic hepatitis B, the method comprising administering an HBV therapy to the subject wherein it has been determined that a post-baseline HBV antibody concentration in the subject after being administered an HBsAg is greater than a baseline HBV antibody concentration in the subject prior to being administered the HBsAg.

In some embodiments, the HBV therapy is administered when it has been determined that a post-baseline HBV antibody concentration in the subject after being administered an HBsAg is 5 times greater than a baseline HBV antibody concentration in the subject prior to being administered the HBsAg.

In some embodiments, the HBV therapy is administered to the subject wherein (i) the subject's baseline HBV antibody concentration is less than or equal to a lower limit of detection (e.g., ≤2 IU/L, and the subject's post-baseline HBV antibody concentration is greater than the lower limit of detection (e.g., >2 IU/L), or (ii) the subject's baseline HBV antibody concentration greater than the lower limit of detection (e.g., >2 IU/L), and the subject's post-baseline HBV antibody concentration is at least 2 times greater than the subject's baseline HBV antibody concentration.

In some embodiments, the baseline and post-baseline HBV antibody concentrations are anti-HBs concentrations determined from serum samples collected from the subject. In some embodiments, the HBV therapy excludes the HBsAg. In some embodiments, the HBV therapy is an siRNA, interferon alfa (IFNα), pegylated interferon alfa (PEG-IFNα), an HBV-neutralizing mAb, or a combination thereof.

In one aspect, provided herein is a method for treating an HBV infection in a subject diagnosed with, or suspected of having an HBV infection, the method comprising: (a) administering an HBsAg to the subject; (b) determining a post-baseline HBV antibody concentration in a serum sample collected from the subject after being administered with the HBsAg; and (c) administering an HBV therapy to the subject if the post-baseline HBV antibody concentration is greater than a baseline HBV antibody concentration in a serum sample collected from the subject prior to being administered with the HBsAg.

In some embodiments, the HBV therapy is administered when it has been determined that a post-baseline HBV antibody concentration in the subject after being administered an HBsAg is 5 times greater than a baseline HBV antibody concentration in the subject prior to being administered the HBsAg.

In some embodiments, the HBV therapy is administered to the subject if (i) the subject's baseline HBV antibody concentration is less than or equal to a lower limit of detection (e.g., ≤2 IU/L), and the subject's post-baseline HBV antibody concentration is greater than the lower limit of detection (e.g., >2 IU/L), or (ii) the subject's baseline HBV antibody concentration greater than the lower limit of detection (e.g., >2 IU/L), and the subject's post-baseline HBV antibody concentration is at least 2 times greater than the subject's baseline HBV antibody concentration. In some embodiments, the baseline and post-baseline HBV antibody concentrations are anti-HBs concentrations. In some embodiments, the subject is virally suppressed with a NrtI prior to administration of the HBsAg. In some embodiments, the HBV therapy excludes the HBsAg. In some embodiments, the HBV therapy is an siRNA, interferon alfa (IFNα), pegylated interferon alfa (PEG-IFNα), an HBV-neutralizing mAb, or a combination thereof.

In one aspect, provided herein is a method for treating an HBV infection in a subject with chronic hepatitis B wherein the HBV antibody concentration in the subject is below 2 IU/L, the method comprising: (a) administering an HBsAg to the subject; (b) administering an HBV therapy to the subject wherein it has been determined that the HBV antibody concentration in the subject is at least 2 IU/L, at least 5 IU/L, at least 10 IU/L, at least 15 IU/L, at least 20 IU/L, at least 25 IU/L, at least 30 IU/L, at least 40 IU/L, at least 50 IU/L, at least 60 IU/L, at least 70 IU/L, at least 80 IU/L, at least 90 IU/L, at least 100 IU/L, at least 200 IU/L, at least 300 IU/L, at least 400 IU/L, at least 500 IU/L, at least 600 IU/L, at least 700 IU/L, at least 800 IU/L, at least 900 IU/L, or at least 1000 IU/L after administration of the HBsAg. In some embodiments, the subject is virally suppressed with a NrtI prior to administration of the HBsAg. In some embodiments, the HBV antibodies are anti-HBs. In some embodiments, the HBV therapy excludes the HBsAg. In some embodiments, the HBV therapy is an siRNA, interferon alfa (IFNα), pegylated interferon alfa (PEG-IFNα), an HBV-neutralizing mAb, or a combination thereof.

In one aspect, provided herein is a method for treating an HBV infection in a subject with chronic hepatitis B, the method comprising administering a therapeutically effective amount of an HBV therapy to a subject with chronic hepatitis B wherein, prior to administering the HBV therapy, it has been determined that the subject's HBV antibody concentration is at least 10 IU/L, at least 15 IU/L, at least 20 IU/L, at least 25 IU/L, at least 30 IU/L, at least 40 IU/L, at least 50 IU/L, at least 60 IU/L, at least 70 IU/L, at least 80 IU/L, at least 90 IU/L, at least 100 IU/L, at least 200 IU/L, at least 300 IU/L, at least 400 IU/L, at least 500 IU/L, at least 600 IU/L, at least 700 IU/L, at least 800 IU/L, at least 900 IU/L, or at least 1000 IU/L. In some embodiments, the subject's HBV antibody concentration is an anti-HBs concentration. In some embodiments, the HBV therapy excludes an HBsAg. In some embodiments, the HBV therapy is an siRNA, interferon alfa (IFNα), pegylated interferon alfa (PEG-IFNα), an HBV-neutralizing mAb, or a combination thereof.

In one aspect, provided herein is an HBV surface antigen (HBsAg) for use in a method as provided herein.

In another aspect, provided herein is a use of an HBV surface antigen (HBsAg) and an HBV therapy not comprising the HBsAg for the manufacture of a kit for treating a subject with chronic hepatitis B who, prior to the administration of the HBV therapy, responds with an increase in an HBV antibody concentration upon being administered with the HBsAg relative to the subject's HBV antibody concentration prior to administration of the HBsAg to the subject. In some embodiments, (i) the subject's HBV antibody concentration prior to administration of the HBsAg to the subject is less than 2 IU/L, or (ii) the subject responds with an increase in HBV antibody concentration that is 2 times greater than the HBV antibody concentration prior to administration of the HBsAg. In some embodiments, the subject is virally suppressed with a NrtI prior to administration of the HBsAg. In some embodiments, the HBV antibodies are anti-HBs. In some embodiments, the HBV therapy is an siRNA, interferon alfa (IFNα), pegylated interferon alfa (PEG-IFNα), an HBV-neutralizing mAb, or a combination thereof.

### BRIEF DESCRIPTION OF THE DRAWINGS

FIG. 1 is a schema of a study design as discussed in Example 8.

FIG. 2 shows the changes of HBsAg levels in patients as compared to baseline, following treatments.

FIG. 3 shows the anti-HBs antibody responses following the treatments. One participant withdrew from the study prior to work 8 and did not have available post baseline anti-HBs data.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

It is to be further understood that wherever aspects are described herein with the language “comprising,” otherwise analogous aspects described in terms of “consisting of” and/or “consisting essentially of” are also provided.

A “therapeutically effective amount,” “effective dose,” “effective amount,” or “therapeutically effective dosage” of a therapeutic agent, e.g., anti-HBsAg siRNA, etc., is any amount that, when used alone or in combination with another therapeutic agent, protects a subject against the onset of a disease or symptom derived from CHB, including for example, liver failure or liver cancer, or promotes disease regression evidenced by a reduction of the viral load, as determined, e.g., by measuring HBV DNA level or by measuring HBV antigen level, a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By “subject” or “individual” or “animal” or “patient” or “mammal,” is meant any subject, particularly a mammalian subject, for whom diagnosis, prognosis, or therapy is desired. In some embodiments, the subject refers to a human. In some embodiments, the subject is a human with chronic hepatitis B (CHB) (“CHB subject”).

As used herein, phrases such as “to a patient in need of treatment” or “a subject in need of treatment” includes subjects, such as mammalian subjects, that would benefit from administration of a composition of the present disclosure used, e.g., for detection, for a diagnostic procedure and/or for treatment.

### Assessment and Treatment of Chronic HBV

It is hereby discovered that BRII-179, a virus-like particle (VLP) that contains HBV 3 surface envelope proteins (L, M, S), induced significant anti-HBs (hepatitis B surface antibody) responses in some but not all participants with chronic HBV (cHBV) infection (Example 1). Moreover, potent anti-HBs responses have been associated with the sustainability of HBsAg (hepatitis B surface antigen) seroclearance following treatment with a BRII-835 and PEG-IFNα combination therapy and a BRII-179 and PEG-IFNα add-on therapy in a subset of CHB patients.

BRII-179, also known as VBI-2601, is a VLP-based recombinant protein immunotherapeutic consisting of three HBsAg forms: small, middle and large proteins that form the HBV envelope. The large S protein contains the N-terminal preS1 and preS2 domains and the adjacent small S protein. The middle S protein contains preS2 and the adjacent small S protein. The preS1 domain contains several relatively conserved B and T helper cell epitopes. Therefore, BRII-179 utilizes PreS1, PreS2, and S protein components as the immunogen in a therapeutic application of preS1/S2 containing HBsAg. In addition, BRII-179 utilizes an aluminum phosphate adjuvant that results in enhanced Th 1-type T cell and humoral immunity.

BRII-835, also known as clebsiran and as VIR-2218, is a synthetic ribonucleic acid interference (RNAi) therapeutic, designed to target HBV transcripts containing a region in the HBx gene that is common to all HBV transcripts and highly conserved among HBV genotypes. It is conjugated to an N-acetyl galactosamine (GalNAc) ligand to facilitate the delivery to hepatocytes via the asialoglycoprotein receptor (ASGPR).

Interferon α (IFNα) is used for treating hepatitis B by inducing interferon stimulated genes at various points of the HBV lifecycle through different pathways that cause an increase in breakdown in viral RNAs and protection against viral injury. IFNα also stimulates cell-mediated immune responses which target infected hepatocytes leading to a decrease in cells that harbor the intrahepatic HBV cccDNA (covalently closed circular DNA) molecules responsible for persistence of HBV infection. Additionally, it may reshape the immune landscape to induce adaptive immunity by coordinating various immune cells, including NK cells, macrophages, DCs and T cells. Pegylation of IFNα (PEG-IFNα) allows weekly injection. PEG-IFNα is associated with several side-effects including influenza-like symptoms, neutropenia, thrombocytopenia, depression, and less commonly exacerbation or unmasking of autoimmune illnesses as well as hepatitis flares. The main advantages of PEG-IFNα in CHB treatment are finite treatment duration, the absence of resistance, and the potential for immune-mediated control of HBV infection with an opportunity to obtain a sustained virological response off-treatment, especially a chance of HBsAg seroclearance in a subset of CHB patients. However, functional cure after PEG-IFNα monotherapy is only confined in a small percentage of CHB patients (<10%).

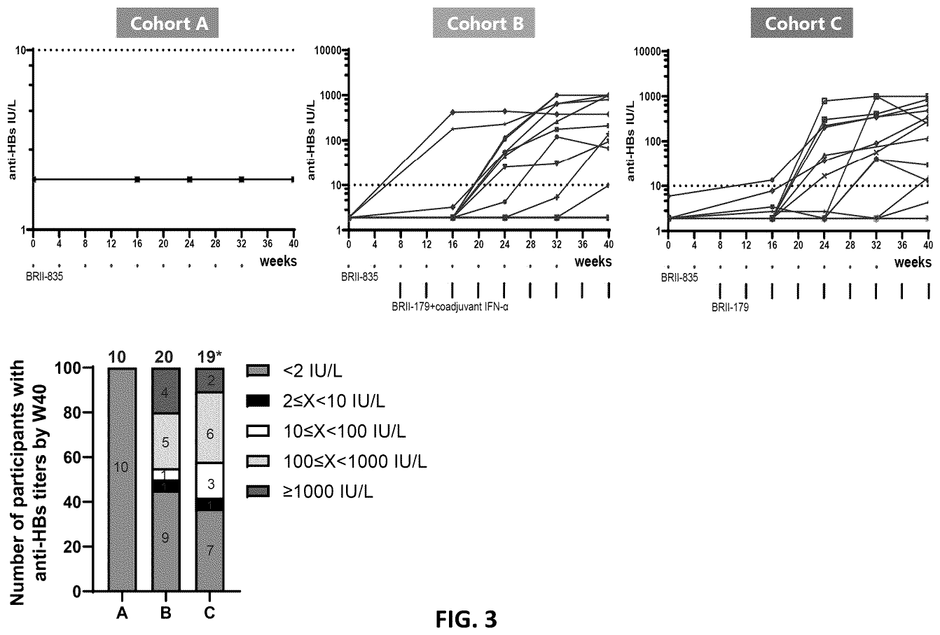

The effect of the addition of BRII-179, as a therapeutic agent to other HBV therapies, was evaluated. As shown in Example 7, BRII-835 alone restored HBV surface-antigen-specific T-cell responses in a small percentage (20%) of participants. The addition of BRII-179 with or without coadjuvant IFN-α induced improved HBV surface-antigen-specific T-cell responses (70%). Further, the combination of BRII-179 and BRII-835 resulted in a higher proportion of participants with greater magnitude of T cell responses (>20-fold of baseline) compared to BRII-179 monotherapy (40% vs 25%). With respect to antibody response, BRII-835 alone in Cohort A did not induce antibody response (FIG. 3). BRII-179 with or without coadjuvant IFN-α induced potent anti-HBs response, generally peaking after 5 doses with titers reaching the upper limit of the assay at 1000 IU/L in some participants. More than 40% of participants in Cohorts B and C mounted high anti-HBs titers (>100 IU/L) by Week 40.

In certain embodiments, for a combination treatment that includes BRII-179 and one or more other HBV therapies, the administration of BRII-179 can commence before the other therapies. It is contemplated that such a treatment schedule not only can take advantage of the synergism between the therapies, but can also allow a patient's response to BRII-179 to serve as a biomarker to guide subsequent therapies.

In one aspect, provided herein are methods for treating an HBV infection in a subject diagnosed with, or suspected of having an HBV infection.

In some embodiments, provided herein is a method for treating CHB in a subject in need thereof comprising administering to the subject a hepatitis B virus surface antigen (HBsAg) and an HBV therapy to the subject. In some embodiments, the HBV therapy excludes the administration of HBsAg to the subject.

In some embodiments, at least a first dose of the HBsAg is administered to the subject before the HBV therapy is administered. In some embodiments, the administration of the HBV therapy does not start until after a second dose of the HBsAg. In some embodiments, the administration of the HBV therapy does not start until after a third dose of the HBsAg. In some embodiments, the administration of the HBV therapy does not start until after a fourth dose of the HBsAg. In some embodiments, the administration of the HBV therapy does not start until after a fifth dose of the HBsAg. In some embodiments, the administration of the HBV therapy does not start until the administration of the HBsAg is completed.

In some embodiments, the administration of the HBV therapy does not start until a week after the initial dose of the HBsAg. In some embodiments, the administration of the HBV therapy does not start until two weeks after the initial dose of the HBsAg. In some embodiments, the administration of the HBV therapy does not start until at least 3, 4, 5, 6, 7, 8, 9, 10, 11, 12, 13, 14, 15, 16, 17, 18, 19, 20 or 21 weeks after the initial dose of the HBsAg.

In some embodiments, the subject being treated herein is treatment-naïve prior to HBsAg administration. In other words, the subject has not treated with a therapy for CHB. In some embodiments, the subject has not been treated with a therapy such as interferons. In some embodiments, the subject has not been treated with at least one of the HBV therapies that are administered following the HBsAg administration (e.g., anti-HBsAg siRNA). In some embodiments, the subject has not been treated with either an interferon or an anti-HBsAg siRNA.

In some embodiments, the subject has not been treated with a nucleoside/nucleotide reverse transcriptase inhibitor (NrtI) prior to HBsAg administration. In some embodiments, the subject has been treated with an NrtI or is being treated with an NrtI.

In some embodiments, provided herein is a method for treating a hepatitis B viral (HBV) infection in a subject with chronic hepatitis B which method comprises administering to the subject 20 μg to 100 μg per dose of an HBV surface antigen (HBsAg) once every two weeks (Q2W), once every three weeks (Q3W) or once every four weeks (Q4W) and 100 mg to 400 mg per dose of an anti-HBsAg siRNA once every two weeks (Q2W), once every three weeks (Q3W), once every four weeks (Q4W), once every five weeks (Q5W) or once every six weeks (Q6W). In some embodiments, the treatment comprises at least four doses for each of the HBsAg and the siRNA. In some embodiments, the treatment comprises at least five, six, seven, eight, nine or ten doses for each of the HBsAg and the siRNA.

In some embodiments, provided herein is a method comprising administering a hepatitis B virus (HBV) therapy to a CHB subject wherein it has been determined that a post-baseline HBV antibody concentration in the CHB subject after being administered a hepatitis B virus surface antigen (HBsAg) is greater than a baseline HBV antibody concentration in the CHB subject prior to being administered the HBsAg.

In some embodiments, provided herein is a method for selecting a CHB subject for a HBV therapy comprising: (a) determining a baseline HBV antibody concentration in a biological sample from the CHB subject; (b) administering an HBsAg to the CHB subject; (c) determining a post-baseline HBV antibody concentration in a biological sample from the CHB subject after being administered with the HBsAg, wherein the subject is selected for the HBV therapy if the post-baseline HBV antibody concentration is greater than the baseline HBV antibody concentration, otherwise the subject is excluded from the HBV therapy; and (d) administering the HBV therapy to the selected subject.

In some embodiments the method comprises: (a) administering an HBsAg to a CHB subject; (b) determining an anti-HBs concentration in a biological sample collected from the subject after being administered with the HBsAg; and (c) administering an HBV therapy to the subject if the anti-HBs concentration in the biological sample collected from the subject after being administered with the HBsAg is greater than a baseline anti-HBs concentration in a biological sample collected from the subject prior to being administered with the HBsAg.

In some embodiments provided herein is a method for treating an HBV infection in a CHB subject wherein a baseline HBV antibody concentration in the CHB subject is below 2 IU/L, the method comprising: (a) administering an HBsAg to the CHB subject; (b) administering an HBV therapy to the CHB subject wherein it has been determined that a post-baseline HBV antibody concentration in the subject is at or above 2 IU/L after administration of the HBsAg.

In some embodiments provided herein is a method for treating an HBV infection in a CHB subject wherein a baseline HBV antibody concentration in the CHB subject is in a range between 0 to 4 IU/L, the method comprising: (a) administering an HBsAg to the CHB subject; (b) administering an HBV therapy to the CHB subject wherein it has been determined that a post-baseline HBV antibody concentration in the subject is above 10 IU/L after administration of the HBsAg.

In some embodiments, the baseline HBV antibody concentration in the CHB subject is in a range between 0 to 10 IU/L.

In some embodiments provided herein is a method for treating an HBV infection in a CHB subject having a predetermined baseline HBV antibody concentration, the method comprising: (a) administering an HBsAg to the CHB subject; (b) administering an HBV therapy to the CHB subject wherein it has been determined that a post-baseline HBV antibody concentration in the subject after administration of the HBsAg is at least 5 times greater than the predetermined baseline HBV antibody concentration.

In one aspect, provided herein are methods for excluding subjects with CHB from a clinical study. In some embodiments, the method comprises screening a group of CHB subjects, wherein each CHB subject has a predetermined baseline HBV antibody concentration, by administering an HBV surface antigen (HBsAg) to the CHB subjects, wherein CHB subjects who do not respond to the HBsAg administration with a post-baseline HBV antibody concentration greater than their baseline HBV antibody concentration are excluded from the clinical study. In certain embodiments, the method further comprises conducting the clinical study wherein an HBV therapy is administered to CHB subjects who are not excluded from the clinical study.

In some embodiments, provided herein is a method for selecting a subject for a clinical study in which an HBV therapy is administered to selected subjects, the method comprising (a) determining a baseline HBV antibody concentration in a subject having CHB prior to administering an HBsAg to the subject, (b) administering the HBsAg to the subject, (c) determining a post-baseline HBV antibody concentration in the subject after administering the HBsAg, wherein the subject is selected for inclusion into a clinical study if the post-baseline HBV antibody concentration is greater than the baseline HBV antibody concentration, and (d) conducting a clinical study wherein an HBV therapy is administered to the selected subject.

The phrase “responder CHB subject” as used herein means a CHB subject who shows an increase in an HBV antibody concentration following administration of an HBsAg to the CHB subject.

The phrase “non-responder CHB subject” as used herein means a CHB subject who does not show an increase in an HBV antibody concentration following administration of an HBsAg to the CHB subject.

Without being limited to any theory or embodiment, it is believed that administration of an HBsAg may induce de novo adaptive immune response and/or boost pre-existing immunological memories specific to surface antigens in responder CHB subjects, thereby achieving greater efficacy of follow-on HBV therapy. Identification of a non-responder CHB subject can, for example, avoid unnecessary costs and personal inconvenience or intolerance of being administered an HBV therapy unlikely to have efficacy for the treatment of CHB in a non-responder CHB subject. Increased HBV antibody response may be a surrogate marker of an intrinsic HBV-specific immunity that may correlate positively with the desired outcome of an HBV therapy; conversely, lack of an HBV antibody response may suggest a weak or absent HBV-specific immunity and correlate negatively with the desired outcome of an HBV therapy.

Diagnosing a subject with CHB typically involves a series of medical tests and assessments. Initially, a medical professional may perform a physical exam to look for signs of liver damage such as jaundice, enlarged liver or spleen, and abdominal swelling. Blood tests are also commonly used to check for the presence of hepatitis B virus DNA, antigens, and antibodies, as well as liver function tests to assess the liver's ability to perform its functions.

In certain embodiments of the methods provided herein, the CHB subject is virally suppressed for HBV, for example, by being on a nucleoside/nucleotide reverse transcriptase inhibitor (NrtI) therapy. The phrase “virally suppressed for HBV” as used herein means, in some embodiments, a serum HBV DNA concentration below a lower limit of detection (which typically varies between about 2 IU/mL to 16 IU/mL depending on the assay system used, see, e.g., Supplementary Table 1 in Kramvis et al., 2022, Nature Reviews Gastroenterology & Hepatology, 19:727-745 and Supplementary Information). In some embodiments, “virally suppressed for HBV” means a serum HBV DNA concentration of less than 100 IU/mL, less than 90 IU/mL, less than 80 IU/mL, less than 70 IU/mL, less than 60 IU/mL, less than 50 IU/mL, less than 40 IU/mL, less than 30 IU/mL, less than 20 IU/mL, less than 15 IU/mL, less than 10 IU/mL, less than 9.6 IU/mL, less than 6.7 IU/mL, less than 2.7 IU/mL, less than 2.4 IU/mL, or less than 2 IU/mL. Test methods for HBV DNA viral load are well known in the art (see, e.g., Abe et al., 1999, J. Clin. Microbiol., 37 (9): 2899-2903; Kramvis et al., 2022, Nature Reviews Gastroenterology & Hepatology, 19:727-745 and Supplementary Information). For example, specimens for HBV DNA viral load can be tested using the Roche COBASE® HBV Quantitative Assay.

In some embodiments, the CHB subject to be administered the HBsAg has been on NrtI therapy. NrtI therapies for treating an HBV infection are known in the art and include, for instance, lamivudine, entecavir, tenofovir alafenamide, tenofovir disoproxil fumarate, adefovir dipivoxil, and telbivudine.

In some embodiments, the CHB subject has been treated with an NrtI and/or an interferon (e.g., interferon alfa (IFNα) or pegylated interferon alfa (PEG-IFNα)) prior to administration of the HBsAg.

In some embodiments, the CHB subject to be administered the HBsAg is not virally suppressed.

In some embodiments of the methods provided herein, the CHB subject has persistent HBsAg in the serum for at least 6 months.

In some embodiments, the CHB subject has an occult HBV infection characterized, for example, by having detectable HBV DNA in a liver specimen and testing negative for serum HBsAg.

It will be understood, in some embodiments of the methods provided herein, the HBsAg is administered to the subject during a diagnostic phase, whereas the HBV therapy is administered to the subject during a treatment phase. During the diagnostic phase, biological samples can be collected from the subject to determine whether the subject responds to HBsAg administration with an increase in an HBV antibody concentration, and, if so, the subject is selected for the treatment phase.

The HBV DNA S gene encodes three envelope/surface proteins in a single open reading frame containing three separate in-frame start codons. This organization defines three protein domains (Pre-S1, Pre-S2, and S) which form the large (“Pre-S1,” having all three domains), medium (“Pre-S2,” having the Pre-S2 and S domains) and small (“S”) surface proteins that share a common C terminus region (sec, e.g., Seitz et al., 2020, Annu. Ref. Virol., 7:263-288). These three proteins form a spherical structure on the surface of the virus, which is highly antigenic, moreover HBV surface protein is typically the first serologic marker to appear in the serum after infection with HBV and is commonly used as a diagnostic test for acute or chronic hepatitis B.

The hepatitis B surface antigen (HBsAg) administered to a subject in the methods provided herein can, for example, comprise any one of large (Pre-S1), medium (Pre-S2) and small(S) HBV surface proteins, and/or portion thereof, or combination thereof. In some embodiments, the HBsAg is a recombinant HBsAg. In some embodiments, the HBsAg is derived from human plasma. In some embodiments, the HBsAg is a virus-like particle. Methods of producing HBsAg from cell culture and producing virus-like particles are known in the art (see, e.g., Wampler et al., 1985, Proc. Natl. Acad. USA, 82 (20): 6830-6834). Moreover, prophylactic HBV vaccines comprising HBsAg have been available for over 30 years, and can, for example, be a source of HBsAg to be administered to the subject in the methods provided herein. Exemplary HBV vaccines include, for instance, HEPLISAV-B (Dynavax Technologies Corporation), which is available in single 0.5 mL doses containing 20 μg HBsAg and 3000 μg CpG 1018 adjuvant; ENGERIX-B (GlaxoSmithKline Biologicals SA), which is available in 0.5 mL (10 μg HBsAg) and 1 mL (20 μg HBsAg) doses with an aluminum hydroxide adjuvant; and PREHEVBRIO (VBI Vaccines), which contains the small(S), middle (Pre-S2) and large (Pre-S1) hepatitis B surface antigens, and is available as a 1 mL (10 μg HBsAg) with an aluminum hydroxide adjuvant.

In some embodiments, the HBsAg consists of the small(S) protein as the only hepatitis B surface antigen.

In some embodiments, the HBsAg administered to the subject contains the small(S), middle (Pre-S2) and large (Pre-S1) proteins and an aluminum phosphate adjuvant. Production and administration of compositions comprising HBsAg and an aluminum phosphate adjuvant are described in WO 2020/099927 A1. In certain embodiments, the HBsAg administered to the subject is in the form of an HBsAg formulation as described in Example 1 of WO 2020/099927 A1, which is incorporated herein by reference for all purposes in its entirety. In some embodiments, the HBsAg administered to the subject is in a composition comprising S protein, Pre-S1 protein and Pre-S2 protein and an aluminum phosphate adjuvant wherein the composition comprises at least 20 μg/ml of HBsAg antigen and the amount of non-adsorbed antigen is at least 30%. In certain embodiments, the HBsAg administered to the subject is in a composition comprising virus-like particles comprising the small(S), middle (Pre-S2) and large (Pre-S1) proteins.

In some embodiments, the HBsAg is administered to the subject in a dose regimen in which HBsAg doses are administered to the subject over the course of days or weeks. In some embodiments, the HBsAg is administered as a single dose, optionally where the dose is split into two or more injections in a single day. A single dose of HBsAg can, for example, contain between 1 μg to 100 μg HBsAg. In some embodiments of the methods provided herein, the amount of HBsAg administered is between 5 to 100 μg, 5 μg to 80 μg, 10 to 100 μg, or 10 to 80 μg HBsAg. In some embodiments the HBsAg administered to the subject is from about 7 μg to about 60 μg, from about 8 μg to about 50 μg, from about 10 μg to about 60 μg, from about 20 μg to about 60 μg, from about 30 μg to about 50 μg, from about 35 μg to about 45 μg, or from about 10 μg to about 40 μg.

In some embodiments of the methods provided herein, the HBsAg is administered as a single dose per day for 2, 3, 4, 5, 6, 7, 8, 9 or more days, which days can, for example, be consecutive, or can, for example, be spaced apart by about 1 day (e.g., every other day), about 7 days (e.g., weekly), about 14 days (e.g., every 2 weeks), about 21 days (e.g., every 3 weeks), about 28 days (e.g., every 4 weeks), about 30 days (e.g., monthly), about 35 days (e.g., every 5 weeks), about 42 days (e.g., every 6 weeks), or about 60 days (e.g., every 2 months) or yearly. In some embodiments, the HBsAg is administered monthly, e.g., for 4 to 5 months, or, e.g., for up to 9 months or longer.

In some embodiments of the methods provided herein, the HBsAg is administered as a single dose of 10 to 100 μg once every 1, 2, 3, 4, or 5 weeks. In some embodiments of the methods provided herein, the HBsAg is administered as a single dose of 20 to 60 μg once every 1, 2, 3, 4, or 5 weeks. In some embodiments of the methods provided herein, the HBsAg is administered as a single dose of 30 to 50 μg once every 1, 2, 3, 4, or 5 weeks. In some embodiments of the methods provided herein, the HBsAg is administered as a single dose of 10 to 100 μg once every 3 weeks. In some embodiments of the methods provided herein, the HBsAg is administered as a single dose of 20 to 60 μg once every 3 weeks. In some embodiments of the methods provided herein, the HBsAg is administered as a single dose of 30 to 50 μg once every 3 weeks. In some embodiments of the methods provided herein, the HBsAg is administered as a single dose of 40 μg once every 3 weeks. In some embodiments of the methods provided herein, the HBsAg is administered as a single dose of 20 μg to 100 μg per dose once every two weeks (Q2W), once every three weeks (Q3W) or once every four weeks (Q4W).

The HBsAg can, for example, be administered intramuscularly or subcutaneously. In some embodiments, the HBsAg is administered with an adjuvant, such as, for example an aluminum hydroxide adjuvant, an CpG 1018 adjuvant, or an aluminum phosphate adjuvant, among others.

It will be understood that typically in the methods provided herein, the HBsAg is administered as a diagnostic to identify a responder CHB subject prior to administration of an HBV therapy to the responder CHB subject. In some embodiments of the methods provided herein, the HBsAg is administered to a subject in absence of any additional therapeutic agent to treat a hepatitis B viral infection.

In some embodiments of the methods provided herein, the HBsAg is administered to the subject concurrently with a NrtI and/or an interferon (e.g., IFN or PEG-IFN) prior to administration of the HBV therapy.

Determining an HBV antibody concentration in a subject can be performed using assays known in the art. HBV antibodies are produced in the immune system in response to the hepatitis B virus and can include, for example, hepatitis B surface antigen antibody (anti-HBs); hepatitis B e antigen antibody (anti-HBe); and hepatitis B core antigen antibody (anti-HBc). Anti-HBc can be found in an IgM subtype, which typically appears during the early phase of acute HBV infection, and an IgG subtype, which persists for life and is a marker of past or ongoing infection.

In some embodiments of the methods provided herein, the HBV antibody comprises anti-HBs, anti-HBe and anti-HBc. Thus, it will be understood that, in some embodiments, an HBV antibody concentration is a concentration of anti-HBs, anti-HBe, and anti-HBe.

In some embodiments of the methods provided herein, the HBV antibody is an anti-HBS. Thus, it will be understood that, in some embodiments, an HBV antibody concentration is an anti-HBs concentration.

An HBV antibody concentration in a subject can be determined in a biological sample collected from the subject. A biological sample can, for example, be a blood sample, a serum sample, or a plasma sample.

In some embodiments, the biological sample is a plasma sample.

In some embodiments the biological sample is a serum sample.

In some embodiments of the methods provided herein wherein an HBV antibody concentration is determined, the HBV antibody concentration is a serum HBV antibody concentration. In some embodiments, the HBV antibody concentration is a serum anti-HBS concentration. In some embodiments, the HBV antibody concentration is a plasma anti-HBS concentration.

Assays and kits for determining HBV antibodies are commercially available in several formats (see, e.g., Supplementary Table 1 in Kramvis et al., 2022, Nature Reviews Gastroenterology & Hepatology, 19:727-745 and Supplementary Information, listing commercially available kits, which is incorporated herein by reference). Enzyme-linked immunosorbent assay (ELISA) kits use an enzyme-based colorimetric or fluorescent detection system to identify the presence of specific antibodies in the biological sample. Rapid diagnostic tests (RDTs) use lateral flow immunoassay technology. Chemiluminescent immunoassay (CLIA) kits utilize chemiluminescent detection methods to measure HBV antibodies. Radioimmunoassay kits use radioactive tracers to measure the binding of HBV antibodies in biological samples. Exemplary commercially available quantitative HBV antibody kits include, for example, DIASOURCE Anti-HBs Elisa (DIASoure ImmunoAssays S.A., Belgium), ELECSYS® HBsAG II quant II immunoassay (Roche Diagnostics), ARCHITECT AUSAB immunoassay (Abbott Laboratories), and VITROS® Anti-HBs assay (Ortho-Clinical Diagnostics).

Depending upon the particular kit or assay employed, the lower limit of detection (LLOD) of HBV antibody concentration (e.g., anti-HBs concentration) can range from 1 IU/L to 5 IU/L. Thus, in some embodiments, the phrase “lower limit of detection” or “LLOD” as used herein in reference to HBV antibody concentration (e.g., anti-HBs concentration) is 2 IU/L.

As used herein, the term “baseline” describing an antibody concentration (e.g., “baseline HBV antibody concentration,” “baseline anti-HBs concentration,” “baseline plasma HBV antibody concentration,” “baseline serum HBV antibody concentration,” “baseline plasma anti-HBs concentration,” “baseline serum anti-HBs concentration,” and the like) refers to an antibody concentration determined from a biological sample collected from the subject prior to when the HBsAg is administered to the subject.

As used herein, the term “post-baseline” describing an antibody concentration (e.g., “post-baseline HBV antibody concentration,” “post-baseline anti-HBs concentration,” “post-baseline plasma HBV antibody concentration,” “post-baseline serum HBV antibody concentration,” “post-baseline plasma anti-HBs concentration,” “post-baseline serum anti-HBS concentration,” and the like) refers to an antibody concentration determined from a biological sample collected from the subject after HBsAg has been administered to the subject.

In some embodiments of the methods provided herein, the HBV therapy is administered to a subject who has an increase in HBV antibody concentration (e.g., anti-HBs concentration) after administration of HBsAg.

In some embodiments, the HBV therapy is administered to a subject having a post-baseline HBV antibody concentration (e.g., anti-HBs concentration) greater than 10 IU/L, greater than 50 IU/L, greater than 100 IU/L, greater than 150 IU/L, greater than 200 IU/L, greater than 250 IU/L, or greater than 300 IU/L.

In some embodiments of the methods provided herein, an HBV therapy is administered to the subject after determining that a post-baseline HBV antibody concentration (e.g., anti-HBS concentration) is greater than a baseline HBV antibody concentration (e.g., anti-HBS concentration) for the subject.

In some embodiments of the methods provided herein, the HBV antibody concentration (e.g., anti-HBs concentration) in the subject prior to administration of HBsAg can be undetectable. In some embodiments, the subject's baseline HBV antibody concentration is less than 0.5 IU/L. In some embodiments, the subject's baseline HBV antibody concentration is less than 1 IU/L. In some embodiments, the subject's baseline HBV antibody concentration is less than 2 IU/L. In some embodiments, the subject's baseline HBV antibody concentration is less than 3 IU/L. In some embodiments, the subject's baseline HBV antibody concentration is less than 4 IU/L. In some embodiments, the subject's baseline HBV antibody concentration is less than 5 IU/L. In some embodiments, the subject's baseline HBV antibody concentration is less than 6 IU/L. In some embodiments, the subject's baseline HBV antibody concentration is less than 7 IU/L. In some embodiments, the subject's baseline HBV antibody concentration is less than 8 IU/L. In some embodiments, the subject's baseline HBV antibody concentration is less than 9 IU/L. In some embodiments, the subject's baseline HBV antibody concentration is less than 10 IU/L. In some embodiments, the subject's baseline HBV antibody concentration is less than 11 IU/L. In some embodiments, the subject's baseline HBV antibody concentration is less than 12 IU/L. In some embodiments, the subject's baseline HBV antibody concentration is less than 13 IU/L. In some embodiments, the subject's baseline HBV antibody concentration is less than 14 IU/L. In some embodiments, the subject's baseline HBV antibody concentration is less than 15 IU/L. In some embodiments, the subject's baseline HBV antibody concentration is less than 20 IU/L. In some embodiments, the subject's baseline HBV antibody concentration is less than 30 IU/L. In some embodiments, the subject's baseline HBV antibody concentration is less than 40 IU/L. In some embodiments, the subject's baseline HBV antibody concentration is less than 50 IU/L. In some embodiments, the subject's baseline HBV antibody concentration is less than 60 IU/L. In some embodiments, the subject's baseline HBV antibody concentration is less than 75 IU/L. In some embodiments, the subject's baseline HBV antibody concentration is less than 100 IU/L.

In some embodiments of the methods provided herein, the HBV antibody concentration (e.g., anti-HBs concentration) in the subject prior to administration of HBsAg can be between from 0 IU/L to 5 IU/L, from 2 IU/L to 10 IU/L. In some embodiments, the subject's baseline HBV antibody concentration is greater than 10 IU/L, greater than 25 IU/L, greater than 50 IU/L, greater than 100 IU/L, or greater than 200 IU/L.

In some embodiments of the methods provided herein, the HBV therapy is administered to the subject if (i) the subject's baseline HBV antibody concentration (e.g., anti-HBS concentration) is at or below a lower limit of detection (e.g., 2 IU/L), and the subject's post-baseline HBV antibody concentration (e.g., anti-HBs concentration) is above the lower limit of detection (e.g., 2 IU/L); or (ii) the subject's baseline HBV antibody concentration (e.g., anti-HBS concentration) is greater than the lower limit of detection (e.g., 2 IU/L), and the subject's post-baseline HBV antibody concentration (e.g., anti-HBs concentration) is at least 5 time greater than the subject's baseline HBV antibody concentration (e.g., anti-HBs concentration).

In some embodiments of the methods provided herein wherein a subject's baseline HBV antibody concentration (e.g., anti-HBs concentration) is below a lower limit of detection (e.g., 2 IU/L), the HBV therapy is administered to the subject if the subject's post-baseline HBV antibody concentration (e.g., anti-HBs concentration) is at least 2 IU/L, at least 5 IU/L, at least 10 IU/L, at least 15 IU/L, at least 20 IU/L, at least 25 IU/L, at least 30 IU/L, at least 40 IU/L, at least 50 IU/L, at least 60 IU/L, at least 70 IU/L, at least 80 IU/L, at least 90 IU/L, at least 100 IU/L, at least 200 IU/L, at least 300 IU/L, at least 400 IU/L, at least 500 IU/L, at least 600 IU/L, at least 700 IU/L, at least 800 IU/L, at least 900 IU/L, or at least 1000 IU/L.

In some embodiments of the methods provided herein wherein a subject's baseline HBV antibody concentration (e.g., anti-HBs concentration) is above a lower limit of detection (e.g., 2 IU/L), the HBV therapy is administered to the subject if the subject's post-baseline HBV antibody concentration (e.g., anti-HBs concentration) is at least 2 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 3 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 4 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 5 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 6 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 7 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 8 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 9 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 10 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 11 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 12 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 13 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 14 times greater than the subject's baseline HBV antibody concentration (e.g., anti-HBs concentration). In some embodiments, the HBV therapy is administered to the subject if the subject's post-baseline HBV antibody concentration (e.g., anti-HBs concentration) is at least 15 times greater than the subject's baseline HBV antibody concentration (e.g., anti-HBs concentration).

In some embodiments of the methods provided herein, the HBV therapy is administered to a subject identified as being a “responder subject” during the diagnostic phase by meeting any one or more of the following criteria (A) to (C): (A) having a baseline HBV antibody concentration (e.g., anti-HBs concentration) that is at or below a lower limit of detection (e.g., ≤2 IU/L), and a post-baseline HBV antibody concentration (e.g., anti-HBs concentration) of at least 5 IU/L, or at least 10 IU/L, at least 12 IU/L, at least 15 IU/L, at least 20 IU/L, at least 30 IU/L, at least 40 IU/L, at least 50 IU/L, at least 60 IU/L, at least 70 IU/L, at least 80 IU/L, at least 90 IU/L, at least 100 IU/L, or at least 110 IU/L; (B) having a baseline HBV antibody concentration (e.g., anti-HBs concentration) that is above a lower limit of detection (e.g., >2 IU/L), and a post-baseline HBV antibody concentration (e.g., anti-HBs concentration) that is at least 2 times, at least 3 times, at least 4 times, at least 5 times, at least 6 times, or at least 7 times greater than the subject's baseline HBV antibody concentration; or (C) having a post-baseline HBV antibody concentration (e.g., anti-HBs concentration) that is at least 10 IU/L, at least 12 IU/L, at least 15 IU/L, at least 20 IU/L, at least 30 IU/L, at least 40 IU/L, at least 50 IU/L, at least 60 IU/L, at least 70 IU/L, at least 80 IU/L, at least 90 IU/L, at least 100 IU/L, or at least 110 IU/L.

Biological samples collected after HBsAg administration to determine an HBV antibody concentration (e.g., anti-HBs concentration), can be collected at any time during the diagnostic phase, for example, after a first dose of HBsAg is administered (e.g., where a single dose is administered), and/or between doses of HBsAg (where more than one dose is administered), and/or after a last dose of HBsAg is administered (where more than one dose is administered).

Samples collected after HBsAg administration to determine an HBV antibody concentration (e.g., anti-HBs concentration), can, for example, be collected anytime between 1 day after HBsAg administration to about 12 months after HBsAg administration. In some embodiments, the post-base HBV antibody concentration is determined from a sample collected 2 months to 8 months after HBsAg administration. In some embodiments, the post-base anti-HBV antibody concentration is determined from a sample collected 3 months to 6 months after HBsAg administration. In some embodiments, the post-base HBV antibody concentration is determined from a sample collected two weeks to 6 months after HBsAg administration. In some embodiments, the post-base HBV antibody concentration is determined from a sample collected 5 days to 4 months after HBsAg administration. In some embodiments, the post-base HBV antibody concentration is determined from a sample collected 7 days to 3 months after HBsAg administration. In some embodiments, the post-base HBV antibody concentration is determined from a sample collected 7 days to 2 months after HBsAg administration. In some embodiments, the post-base HBV antibody concentration is determined from a sample collected two weeks to 1 month after HBsAg administration.

The HBV therapy, in some embodiments, is administered to a responder CHB subject in a therapeutically effective amount to treat CHB.

The terms “treating,” and “treatment” as used herein refer to both treatment of an existing HBV infection (e.g., CHB) or prevention (i.e., prophylaxis) of severe liver diseases such as liver cirrhosis and hepatocellular carcinoma caused by CHB. It will be therefore recognized that treating or treatment as referred to herein may, in some embodiments, be prophylactic. In some embodiments, treating or treatment of an HBV infection (e.g., CHB) results in a reduction of HBV DNA, or a reduction for hepatitis B e antigen, or a reduction of serum alanine aminotransferase (ALT), or a reduction of hepatitis B's antigen, or any combination thereof. In some embodiments, treating or treatment of an HBV infection can be a functional cure where HBsAg and HBV DNA are undetectable in a subject for 6 months in the absence of any HBV therapy. The phrase “therapeutically effective amount” as used herein refers to an amount of a therapy, alone or in combination with other therapies, for treating an HBV infection, as “treating” is defined above.

The HBV therapy that is administered to the subject can be any therapy administered for treatment of an HBV infection, e.g., CHB.

In some embodiments, the HBV therapy is administered in a clinical study.

In some embodiments, the HBV therapy is single therapy, or a combination of therapies, selected from the following HBV therapies: (i) a nucleoside/nucleotide reverse transcriptase inhibitor (NrtI), such as, e.g., lamivudine, entecavir, tenofovir alafenamide, tenofovir disoproxil fumarate, adefovir dipivoxil, and telbivudine; (ii) an immunomodulator, such as, e.g., interferon alfa (IFNα) and pegylated interferon alfa (PEG-IFNα); (iii) a small interfering RNA (siRNA) targeting a HBV RNA, such as, e.g., AB-729, ALG-125755, ALG-125918, ARB-1467, ARC-520, JNJ-3989, RG-6346, VIR-2218 (BRII-835 or elebsiran), and those described in WO 2016/077321 A1 and WO 2020/036862 A1; (iv) an HBV-neutralizing monoclonal antibody (mAb), such as, e.g., those described in WO/2017/060504 A1, WO 2020/132091 A2, WO 2022/164805 A1, WO 2021/012135 A1, U.S. Pat. No. 10,544,205 B2, and including, for example, VIR-3434 (BRII-877), HH-003, HH-006, BJT-778, and lenvervimab (GC1102); (v) a sodium/bile acid cotransporter (also known as NTCP) inhibitor, such as, e.g., myrcludex B (bulevirtide); (vi) a capsid assembly modulator, such as, e.g., ABI-HO731, ABI-H3733, NVR 3-778, RO7049389, JNJ-56136389, ALG-000184, and GLS4JHS; (vii) a COPS3 inhibitor, such as, e.g., those as described in U.S. Pat. No. 20,230,122751 A1; (viii) a store-operated calcium entry-associated regulatory factor (SARAF) inhibitor, such as, e.g., those as described in US 2023/0120063 A1; (ix) an antisense oligonucleotide (ASO), such as, e.g., bepirovirsen, GSK3389404, RO7062931, and ALG-020572; (x) a Toll-like receptor (TLR) agonist, such as, e.g., vestolimod (GS-9620; TLR7 agonist), RO7020531 (TLR7 agonist), JNJ-64794964 (TLR7 agonist), selgantolimod (GS-9688; TLR8 agonist), and AIC649 (TLR9 agonist); (xi) a checkpoint modulator, such as, e.g., ASC22 (anti-PDL1), cemiplimab (anti-PD1), and nivolumab (anti-PD1); (xii) an inhibitor of apoptosis protein (IAP) antagonist, such as, e.g., APG-1387; (xiii) a farnesoid X receptor (FXR) agonist, such as, e.g., EYP001; and (xiv) a therapeutic vaccine, such as, e.g., ABX203 (HeberNasvac), GS-4774, HepTcell, AIC649, HB-110, VTP-300, JNJ-64300535, BRII-179 (VBI-2601), TG-1050, and INO-1800.

In some embodiments, the HBV therapy is selected from the group consisting of a NrtI, siRNA, IFNα, PEG-IFNα, and an HBV-neutralizing mAb, or a combination thereof. In some embodiments, the HBV therapy is selected from the group consisting of an siRNA, IFNα, and PEG-IFNα, an HBV-neutralizing mAb, or a combination thereof. In some embodiments, the HBV therapy is selected from the group consisting of an siRNA, IFNα, and PEG-IFNα, or a combination thereof. In some embodiments, the HBV therapy is an siRNA. In some embodiments, the HBV therapy is a combination of an siRNA and IFNα. In some embodiments, the HBV therapy is a combination of an siRNA and PEG-IFNα. In some embodiments, the HBV therapy is a combination of an HBV-neutralizing mAb and PEG-IFNα. In some embodiments, the HBV therapy is PEG-IFNα.

In some embodiments, the HBV therapy is a combination of a NrtI and interferon (e.g., IFNα or PEG-IFNα).

In some embodiments, the HBV therapy is administered in combination with a HBsAg. In some embodiments, the HBV therapy excludes HBsAg.

In some embodiments in which an siRNA is administered alone or in a combination HBV therapy, the siRNA is a N-acetylgalactosamine (GalNAc)-conjugated HBV dsRNA as described in Table 2 of WO 2020/036862 A1, which is incorporated herein by reference for all purposes. In some embodiments, the siRNA is Elebsiran (CAS No. 2648009-64-5) (also known as VIR-2218 and BRII-835).

In some embodiments in which an HBV-neutralizing antibody is administered alone in a combination, the HBV-neutralizing antibody is VIR-3434 (BRII-877).

In some embodiments, the HBV therapy is a combination of an siRNA and an HBV-neutralizing antibody. In some embodiments, the HBV therapy is a combination of an siRNA, an HBV-neutralizing antibody and PEG-IFNα. For instance, in some embodiments, the siRNA is Elebsiran (CAS No. 2648009-64-5; VIR-2218; BRII-835) and the HBV-neutralizing antibody is VIR-3434 (BRII-877).

In some embodiments, the HBV therapy is a combination of Elebsiran (CAS No. 2648009-64-5) and PEG-IFNα.

In some embodiments, the siRNA (e.g., elebsiran) is administered as a single dose per day for 2, 3, 4, 5, 6, 7, 8, 9, 10, 11, 12, 13, 14, 15, 16, 17, 18, 29 or 20, or more days, which days can, for example, be consecutive, or can, for example, be spaced apart by about 1 day (e.g., every other day), about 7 days (e.g., weekly), about 14 days (e.g., every 2 weeks), about 21 days (e.g., every 3 weeks), about 28 days (e.g., every 4 weeks), about 30 days (e.g., monthly), about 35 days (e.g., every 5 weeks), about 42 days (e.g., every 6 weeks), or about 60 days (e.g., every 2 months) or yearly.

In some embodiments of the methods provided herein, the siRNA (e.g., elebsiran) is administered as a single dose of 20 to 500 mg once every 1, 2, 3, 4, 5 or 6 weeks. In some embodiments of the methods provided herein, the siRNA (e.g., elebsiran) is administered as a single dose of 30 to 400 mg once every 1, 2, 3, 4, 5 or 6 weeks. In some embodiments of the methods provided herein, the siRNA (e.g., elebsiran) is administered as a single dose of 40 to 250 mg once every 1, 2, 3, 4, 5 or 6 weeks. In some embodiments of the methods provided herein, the siRNA (e.g., elebsiran) is administered as a single dose of 50 to 200 mg once every 1, 2, 3, 4, 5 or 6 weeks. In some embodiments of the methods provided herein, the siRNA (e.g., elebsiran) is administered as a single dose of 75 to 150 mg once every 1, 2, 3, 4, 5 or 6 weeks. In some embodiments of the methods provided herein, the siRNA (e.g., elebsiran) is administered as a single dose of 80 to 120 mg once every 1, 2, 3, 4, 5 or 6 weeks.

In some embodiments of the methods provided herein, the siRNA (e.g., elebsiran) is administered as a single dose of 50 to 50 mg once every 4 weeks. In some embodiments of the methods provided herein, the siRNA (e.g., elebsiran) is administered as a single dose of 75 to 150 mg once every 4 weeks. In some embodiments of the methods provided herein, the siRNA (e.g., elebsiran) is administered as a single dose of 80 to 120 mg once every 4 weeks. In some embodiments of the methods provided herein, the siRNA (e.g., elebsiran) is administered as a single dose of 90 to 110 mg once every 4 weeks. In some embodiments of the methods provided herein, the siRNA (e.g., elebsiran) is administered as a single dose of about 100 mg once every 4 weeks. In some embodiments of the methods provided herein, the siRNA (e.g., elebsiran) is administered as a single dose of 100 mg to 400 mg per dose once every two weeks (Q2W), once every three weeks (Q3W), once every four weeks (Q4W), once every five weeks (Q5W) or once every six weeks (Q6W).

The siRNA (e.g., elebsiran) can, for example, be administered intramuscularly or subcutaneously.

In some embodiments, the PEG-IFNα is administered as a single dose per day for 2, 3, 4, 5, 6, 7, 8, 9, 10, 20, 30, 40, 45, 48, 50 or 60, or more days, which days can, for example, be consecutive, or can, for example, be spaced apart by about 1 day (e.g., every other day), about 7 days (e.g., weekly), about 14 days (e.g., every 2 weeks), about 21 days (e.g., every 3 weeks), about 28 days (e.g., every 4 weeks), about 30 days (e.g., monthly), about 35 days (e.g., every 5 weeks), about 42 days (e.g., every 6 weeks), or about 60 days (e.g., every 2 months) or yearly.

In some embodiments of the methods provided herein, the PEG-IFNα is administered as a single dose of 50 to 500 μg once every 1, 2 or 3 weeks. In some embodiments of the methods provided herein, the PEG-IFNα is administered as a single dose of 100 to 300 μg once every 1, 2 or 3 weeks. In some embodiments of the methods provided herein, the PEG-IFNα is administered as a single dose of 150 to 240 μg once every 1, 2 or 3 weeks. In some embodiments of the methods provided herein, the PEG-IFNα is administered as a single dose of 170 to 190 μg once every 1, 2 or 3 weeks.

In some embodiments of the methods provided herein, the PEG-IFNα is administered as a single dose of 50 to 500 μg once every week. In some embodiments of the methods provided herein, the PEG-IFNα is administered as a single dose of 100 to 300 μg once every week. In some embodiments of the methods provided herein, the PEG-IFNα is administered as a single dose of 150 to 240 μg once every week. In some embodiments of the methods provided herein, the PEG-IFNα is administered as a single dose of 170 to 190 μg once every week.

The PEG-IFNα can, for example, be administered intramuscularly or subcutaneously.

In one aspect, provided herein are compositions for use in the methods as described herein. In some embodiments, the composition is an HBsAg for use in the methods as described herein. Suitable HBsAg compositions can, for example, be any of those as described herein. In some embodiments, the composition is an HBV therapy for use in the methods as described herein. Suitable HBV therapies can, for example, be any of those as described herein.

In one aspect, provided herein is a kit comprising an HBsAg and an HBV therapy for use in the methods as described herein. Suitable compositions for the HBsAg and the HBV therapy can, for example, be any of those described herein.

In some embodiments, provided herein is a use of an HBV surface antigen (HBsAg) and an HBV therapy not comprising the HBsAg for the manufacture of a kit for treating a subject with chronic hepatitis B who, prior to the administration of the HBV therapy, responds with an increase in serum concentration of anti-HBV antibodies upon being administered with the HBsAg relative to the subject's serum concentration of anti-HBV prior to administration of the HBsAg to the subject.

In some embodiments, the treatment is effective in achieving disease regression in the CHB subject. For instance, disease regression may be measured as a reduction in viral load or HBV antigen seroclearance (e.g., by measuring HBV DNA level or antigen level), without limitation.

In some embodiments, the HBV DNA level or antigen level is reduced at least by about 10%, 20%, 30%, 40%, 50%, 60%, 70%, 80%, 90%, 95%, 98%, or 99%. In some embodiments, the HBV DNA level or antigen level is reduced to a desired level for at least 1 week, 2 weeks, 3 weeks, 4 weeks, 2 months, 3 months, 4 months, 5 months, 6 months, 1 year, 2 years or 5 years.

### EXAMPLES

### Example 1: Responder CHB Subjects

This example demonstrates that CHB subjects differentiate into those producing anti-HBS in response to HBsAg administration (responder CHB subjects) and those that do not show such a response (non-responder CHB subjects).

A clinical study in which an HBsAg was administered to non-cirrhotic CHB subjects virally suppressed under NrtI therapy was conducted as described in Ma et al., 2021, JHEP Reports, 3(3):100361. The HBsAg was BRII-179 (also known as VBI-2601), which is a virus-like particle-containing all 3 (Pre-S1, Pre-S2 and S) HBV surface envelope proteins. Subjects were randomized to receive NrtI only (Cohort A), 20 μg BRII-179 plus NrtI (Cohort B), 20 μg BRII-179 plus 3 MIU IFN plus NrtI (Cohort C), 40 μg BRII-179 plus NrtI (Cohort D), and 40 μg BRII-179 plus 3 MIU IFN plus NrtI (Cohort E). Serum HBsAg and anti-HBs antibodies were quantified with ELECSYS HBsAg II QUANT II kit and ELECSYS ANTI-HBS II kit/Cobas e411/e601 (Roche Diagnostics, Germany), respectfully, according to manufacturer's instructions. The number of subjects observed having a positive response (i.e., an increase in post-baseline antibody concentration) is shown in Table 1 below.

TABLE 1
Anti-HBs Response of BRII-179 in CHB subjects*

| | | |
|---|---|---|
| Cohort | Anti-HBs Responders | Non-responders |

| | | | | |
|---|---|---|---|---|
| A | 0/5 | (0%) | 5/5 | (100%) |
| B | 6/10 | (60%) | 4/10 | (40%) |
| C | 3/9 | (33.3%) | 6/9 | (66.7%) |
| D | 6/12 | (50%) | 6/12 | (50%) |
| E | 4/12 | (33.3%) | 8/12 | (66.7%) |

*Positive anti-HBs antibody (Ab) response was defined at any post-baseline visit with either (i) post-baseline anti-HBsAb ≥2 IU/L if anti-HBsAb was undetectable at baseline or (ii) post-baseline anti-HBsAb ≥5 times the baseline anti-HBsAb if baseline anti-HBsAg was ≥2 IU/L.

### Example 2: Administration of an HBV Therapy to Responder CHB Subjects

This prophetic example demonstrates an exemplary administration of an HBV therapy to responder CHB subjects. Responder CHB subjects identified by having an increase in anti-HBs following administration of an HBsAg (e.g., the patients identified as responders CHB subjects in Example 1) can have an HBV therapy administered as described below.

The HBV therapy can be a combination of siRNA and PEG-IFNα. Specifically, the siRNA can be BRII-835 (also known as elebsiran and as VIR-2218), which is a synthetic N-acetylagalactosamine-conjugated small ribonucleic acid designed to silence HBV transcripts across all HBV genotypes. BRII-835 has been administered in single doses of up 900 mg in healthy volunteers and subjects with chronic HBV infection (see, e.g., AASLD 2022, VIR-2218-1001 study: Preliminary 48-week Safety and Efficacy Data of VIR-2218 Alone and in Combination With Pegylated Interferon Alfa in Participants With Chronic HBV Infection; ClinicalTrials.gov identifier: NCT03672188; and Gupta et al., 2021, Drugs in R&D, 21:455-465).

Key criteria for excluding subjects from receiving the therapy can be as follows: 1. Being administered with other siRNA or interferon-based therapies within 12 months prior to screening. 2. Significant fibrosis or cirrhosis. 3. Contraindications to the use of PEG-IFNα or incapable of self-administration or assisted administration of PEG-IFNα, including but not limited to below laboratory abnormalities: a) Hemoglobin<100 g/L. b) Absolute neutrophil count<1,500/mm3 c) Platelet count<90,000/mm3. 4. Active HIV, HCV, or HDV infection. 5. Renal impairment with estimated glomerular filtration rate (eGFR)≤60 ml/min/1.73 m2 at screening. 6. History of intolerance or hypersensitivity to interferon/pegylated interferon.

The dose and dosage regimen of the HBV therapy administered to the responder CHB subjects can be 100 mg BRII-835 administered by subcutaneous (SC) injection every four weeks, and 180 μg PEG-IFNα administered by SC injection every week, over a period of 48 weeks.

CHB subjects can be monitored for suppression of HBV DNA (<LLOQ) with HBsAg loss (<0.05 IU/mL) to assess effectiveness.

### Example 3: Administration of an HBV Therapy to Responder CHB Subjects

This prophetic example demonstrates an exemplary administration of an HBV therapy to responder CHB subjects in which the HBV therapy comprises an siRNA and PEG-IFNα. The dose and dosage regimen of the HBV therapy administered to the responder CHB subjects can be 200 mg BRII-835 administered by subcutaneous (SC) injection every four weeks, and 180 μg PEG-IFNα administered by SC injection every week, over a period of 48 weeks. CHB subjects can be monitored for suppression of HBV DNA (<LLOQ) with HBsAg loss (<0.05 IU/mL) to assess effectiveness.

### Example 4: Administration of an HBV Therapy to Responder CHB Subjects

This prophetic example demonstrates an exemplary administration of an HBV therapy to responder CHB subjects in which the HBV therapy comprises an HBV-neutralizing mAb and PEG-IFNα. The dose and dosage regimen of the HBV therapy administered to the responder CHB subjects can be 300 mg BRII-877, a HBV-neutralizing antibody also known as VIR-3434, administered once monthly by SC injection and 180 μg PEG-IFNα administered once weekly by SC injection, over a period of 6 or more months. CHB subjects can be monitored for suppression of HBV DNA (<LLOQ) with HBsAg loss (<0.05 IU/mL) to assess effectiveness.

### Example 5: Administration of an HBV Therapy to Responder CHB Subjects

This prophetic example demonstrates an exemplary administration of an HBV therapy to responder CHB subjects in which the HBV therapy comprises a combination of an siRNA, an HBV-neutralizing mAb and PEG-IFNα. The dose and dosage regimen of the HBV therapy administered to the responder CHB subjects can be 100 mg or 200 mg BRII-835 administered once every four weeks by SC injection, 100 mg or 150 mg BRII-877 administered once monthly by SC injection and 180 μg PEG-IFNα administered once weekly by SC injection, over a period of 48 weeks. CHB subjects can be monitored for suppression of HBV DNA (<LLOQ) with HBsAg loss (<0.05 IU/mL) to assess effectiveness.

### Example 6: Administration of an HBV Therapy to Responder CHB Subjects

This prophetic example demonstrates an exemplary administration of an HBV therapy to responder CHB subjects in which the HBV therapy comprises a combination of an siRNA and an HBV-neutralizing mAb. The dose and dosage regimen of the HBV therapy administered to the responder CHB subjects can be 100 mg or 200 mg BRII-835 administered once every four weeks by SC injection, and 100 mg or 150 mg BRII-877 administered once monthly by SC injection, over a period of 48 weeks. CHB subjects can be monitored for suppression of HBV DNA (<LLOQ) with HBsAg loss (<0.05 IU/mL) to assess effectiveness.

### Example 7: Preliminary Safety and Efficacy of Combination of BRII-835 and BRII-179 in Treating Chronic HBV Infection

This example tested the safety and efficacy of a combination therapy that included both BRII-835 and BRII-179 for treating chronic HBV infection, in a phase II trial.

Adult chronic HBV patients on NrtI therapy >12 months with HBV DNA<LLOQ were enrolled. The participants were enrolled in three cohorts. In Cohort A, each patient was administered, while still on NrtI therapy, 100 mg BRII-835 via subcutaneous injection (SC), once every 4 weeks, for 9 times. In Cohort B, while still on NrtI therapy, each patient was administered 100 mg BRII-835, every 4 weeks, for 9 times; starting on the 3^{rd} dose, the patient also received 40 μg BRII-179+coadjuvant 3 MIU IFN-α via intramuscular injection (IM), for 9 times. Cohort C differed from Cohort B in that no IFN-α was administered with the BRII-179.

In all three cohorts, at the last dose of BRII-835 and/or BRII-179, the patient was evaluated with respect to meeting NrtI discontinuation criteria, defined as undetectable HBsAg and HBeAg, alanine aminotransferase<2× upper limit of normal, and HBV DNA<LLOQ. Those that met the NrtI discontinuation criteria were eligible to withdraw from the NrtI therapy, subject to 48-week NrtI discontinuation monitoring. The demographics and baseline characteristics of the participants are shown in Table 2.

TABLE 2
Participants' demographics and baseline characteristics

| | Cohort A | | Cohort B | | Cohort C | |
|---|---|---|---|---|---|---|
| | (N = 11*) | | (N = 20) | | (N = 20) | |
| Mean Age ± SD | 45.9 ± 10.5 | | 47.6 ± 9.1 | | 45.3 ± 9.5 | |
| (years) | | | | | | |
| Male, n (%) | 8 | (72.7%) | 14 | (70.0%) | 15 | (75.0%) |
| Race, n (%) | | | | | | |
| Asian | 11 | (100%) | 18 | (90.0%) | 18 | (90.0%) |
| Black or African | 0 | | 2 | (10.0%) | 1 | (5.0%) |
| American | | | | | | |
| White | 0 | | 0 | | 1 | (5.0%) |
| HBeAg Status at | | | | | | |
| Baseline, n (%) | | | | | | |
| Negative | 9 | (81.8%) | 15 | (75.0%) | 14 | (70.0%) |
| Positive | 2 | (18.2%) | 5 | (25.0%) | 6 | (30.0%) |
| Median (Range) | 387.3 | | 694.7 | | 832.7 | |
| Baseline HBsAg | (145.4, 1222.0) | | (175.8, 6479.0) | | (160.2, 3169.0) | |
| (IU/mL) | | | | | | |
| Mean Baseline log₁₀ | 2.63 ± 0.30 | | 2.97 ± 0.42 | | 2.90 ± 0.35 | |
| HBsAg ± SD (IU/mL) | | | | | | |

| | | | |
|---|---|---|---|
| Mean Baseline | 20.3 ± 11.4 | 21.4 ± 9.5 | 21.6 ± 9.9 |
| ALT ± SD (U/L) | | | |

*One participant withdrew consent prior to study drug administration
ALT, alanine aminotransferase;
HBeAg, hepatitis B virus e antigen;
HBsAg, hepatitis B virus surface antigen;
SD, standard deviation

Safety evaluation results (Table 3) show that BRII-835 alone or in combination with BRII-179±coadjuvant IFN-a were well tolerated. The majority of TEAEs was grade 1 or 2 in severity; none of ≥grade 3 or serious TEAEs were treatment related. The most common TEAEs across cohorts were injection site reactions (56.0%). TEAEs with higher incidence in Cohort B (i.e., headache, fatigue, myalgia, and pyrexia) were consistent with the known side effects of IFN-α.

TABLE 3
Treatment-emergent adverse event (TEAE)

| | Cohort A | | Cohort B | | Cohort C | |
|---|---|---|---|---|---|---|
| | (N = 10) | | (N = 20) | | (N = 20) | |
| Any TEAEs | 10 | (100%) | 19 | (95.0%) | 20 | (100%) |
| Grade 1 TEAEs | 10 | (100%) | 19 | (95.0%) | 17 | (85.0%) |
| Grade 2 TEAEs | 1 | (10.0%) | 7 | (35.0%) | 6 | (30.0%) |
| ≥Grade 3 TEAEs | 0 | | 0 | | 2 | (10.0%) |
| BRII-835 Related TEAEs | 7 | (70.0%) | 13 | (65.0%) | 10 | (50.0%) |

| BRII-179 + IFN-α Related | NA | 17 | (85.0%) | NA | |
|---|---|---|---|---|---|
| TEAEs | | | | | |
| BRII-179 Related TEAEs | NA | NA | | 10 | (50.0%) |
| Serious TEAEs* | 0 | 1 | (5.0%) | 3 | (15.0%) |

| | | | | |
|---|---|---|---|---|
| AEs Leading to Treatment | 0 | 0 | 1 | (5.0%) |
| Discontinuation^{#} | | | | |
| AEs Leading to Study | 0 | 0 | 1 | (5.0%) |
| Discontinuation^{#} | | | | |

NA, not applicable;
TEAE, treatment-emergent adverse event
*One chest pain (non-cardiac) in Cohort B; one each of duodenal ulcer, Ludwig's angina, and chest pain (non-cardiac) in Cohort C
^{#}One participant experiencing duodenal ulcer withdrew study prematurely

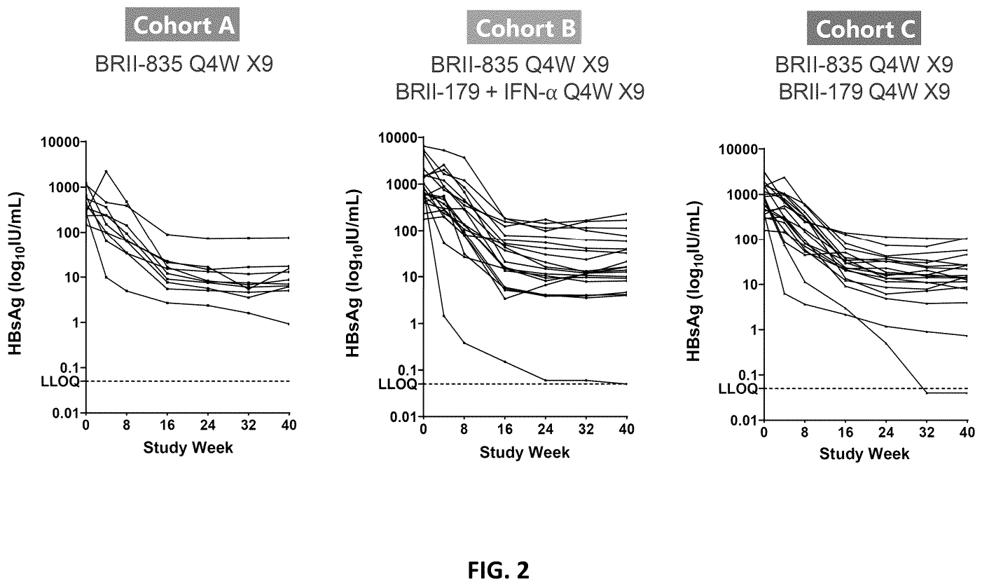

In terms of efficacy, notable HBsAg reductions in the patients were observed with BRII-835 alone or in combination with BRII-179±coadjuvant IFN-α (FIG. 2 and Table 4). Also, the mean HBsAg reductions from baseline were comparable across cohorts. Strikingly, as shown in FIG. 2, two patients that received combination treatments achieved HBsAg≤LLOQ (0.05 IU/mL) by Week 40.

TABLE 4
Mean (SD) HBsAg change from baseline (log₁₀ IU/mL)

| | Week 32 | Week 40 |
|---|---|---|
| | (SIRNA EOT) | (Combo EOT) |
| Cohort A (N = 10) | −1.75 | |
| | (0.39) | |
| Cohort B (N = 20) | −1.78 | −1.75 |
| | (0.58) | (0.60) |
| Cohort C (N = 20) | −1.81 | −1.77 |
| | (0.71) | (0.72) |

With respect to antibody response, BRII-835 alone in Cohort A did not induce antibody response (FIG. 3). BRII-179 induced potent anti-HBs response, generally peaking after 5 doses with titers reaching the upper limit of the assay at 1000 IU/L in some participants. More than 40% of participants in Cohorts B and C mounted high anti-HBs titers (>100 IU/L) by Week 40.

Two early responders with antibody titer peaking after two injections of BRII-179 were only observed in BRII-179+coadjuvant IFN-α group (Cohort B). Combination treatment of 9-doses BRII-179+BRII-835 resulted in a higher percentage of participants with anti-HBs levels above 100 IU/L (44%) compared to BRII-179 monotherapy (4 doses, 17%, FIG. 3).

HBV surface antigen-specific T-cell responses were measured in these patients and the results are shown in Table 5.

TABLE 5
HBV surface antigen-specific T-cell responses

| | Cohort A | Cohort B | Cohort C |
|---|---|---|---|
| In vitro | 1/5 (20%) | 7/10 (70%) | 7/10 (70%) |
| (peptide pools) | | | |

As shown in the table, BRII-835 alone restored HBV surface-antigen-specific T-cell responses in a small percentage of participants. BRII-179 with or without coadjuvant IFN-α induced improved HBV surface-antigen-specific T-cell responses in combination with BRII-835. Comparable HBV surface-antigen-specific T-cell responses were observed in the two combination cohorts receiving BRII-835 and BRII-179 with or without coadjuvant IFN-α.

Importantly, combination treatment of BRII-179+BRII-835 resulted in a higher proportion of participants with greater magnitude of T cell responses (>20-fold of baseline) compared to BRII-179 monotherapy (40% vs 25%).

### Example 8. Phase 2 Multicenter, Open-label Study to Investigate the Efficacy and Safety of Regimens Containing BRII-179, BRII-835, and Pegylated Interferon Alpha (PEG-IFNα) for the Treatment of Chronic Hepatitis B Virus (HBV) Infection

This is a Phase 2 single-arm, open-label study assessing the efficacy and safety of sequential BRII-179 treatment followed by BRII-835 and PEG-IFNα combination therapy in participants with chronic HBV infection.

A preliminary study demonstrated that anti-HBs response were associated with the sustainability of HBsAg seroclearance post EOT in CHB patients receiving BRII-835+PEG-IFNαcombination therapy. In this study, all 4/13 subjects achieved HBsAg seroclearance after 48-week combination therapy have anti-HBs seroconversion. Among participants who had HBsAg seroclearance by EOT, all of those (4/4) with anti-HBs titers>500 IU/L at EOT had sustained HBsAg seroclearance at 24 weeks post EOT; while participants (3/3) with anti-HBS titers<100 IU/L at EOT experienced HBsAg rebound, suggesting high anti-HBs titers at EOT are correlated with the persistence of seroclearance. Additionally, CHB patients with anti-HBS-producing B cells detected in PBMC at baseline may be an important immunological indicator to predict IFNα treatment efficacy, showing a higher chance to reach functional cure to PEG-IFNα treatment. These findings suggested that the capability of mounting humoral immune response to HBsAg in CHB participants may be used to identify potential responders with less compromised endogenous anti-HBV immunity for achieving and maintaining sustained functional cure upon curative regimen treatment.

As shown in Example 1, BRII-179 induced significant anti-HBs responses in some but not all chronic HBV participants even after 9 doses, suggesting that BRII-179 may be used to identify CHB participants who are able to elicit a good antibody response increasing their likelihood of response to BRII-835+PEG-IFNα. Therefore, it is proposed to initiate a clinical study in which CHB participants will receive sequentially BRII-179 followed by BRII-835+PEG-IFNα. Participants will be evaluated whether the predefined criteria of anti-HBS response are met after completion of BRII-179 treatment. This study design will provide an opportunity to compare the efficacy of a finite treatment duration of BRII-835+PEG-IFNα between anti-HBs responders and non-responders after completing BRII-179 treatment and to find an optimal treatment combination with favorable benefit/risk profile.

Data from this study will support further exploration of other curative treatments of finite duration in achieving clinically meaningful functional cure rate for chronic HBV infection.

Enrolled participants will receive sequential BRII-179 followed by BRII-835 and PEG-IFNα combination over a total of 64-week dosing period as the following:
BRII-179 (Day 1 to Week 12): participants will receive 5 doses of BRII-179 40 μg, given at every 3 weeks (Q3W);
BRII-835 and PEG-IFNα combination (Week 16 to Week 64): participants will receive 13 doses of BRII-835 100 mg, given at every 4 weeks (Q4W), and 48 doses of PEG-IFNα 180 μg, given at every week (QW).

There will be a 24-week follow-up period post treatment. Another 24-week NrtI discontinuation monitoring period will apply to participants eligible to discontinue their NrtI therapy.

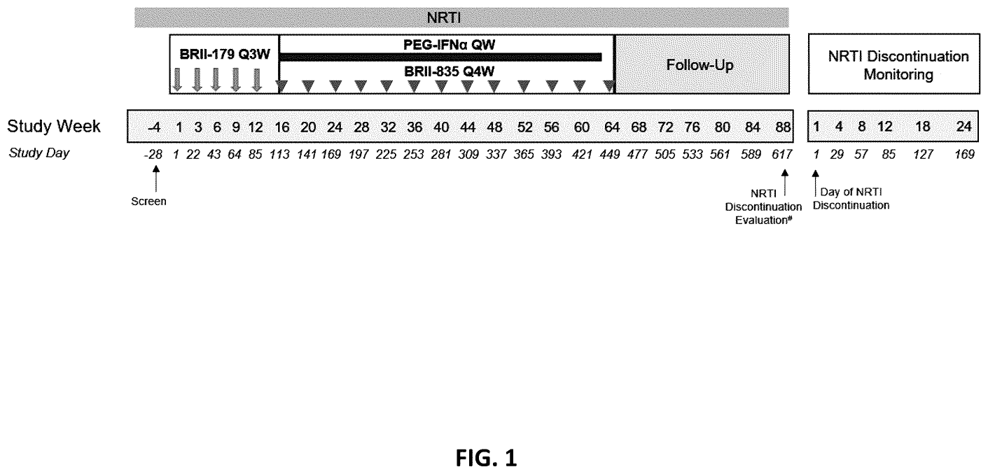

Participants with HBeAg negative or HBeAg positive chronic HBV infection are eligible to participate in this study. A total of 110 participants are planned and another 40 floater participants may be added. The total duration of study participation for each participant will include screening period (up to 4 weeks), dosing period (68 weeks), follow-up period (24 weeks), and NRTI discontinuation monitoring period (24 weeks when applicable). The study design schema is illustrated in FIG. 1.

### Inclusion Criteria

Participants eligible to be included in the study must meet all of the following criteria. All laboratory test requirements for inclusion will be performed by central laboratory unless specified otherwise:
1. A signed and dated written Informed Consent Form (ICF) prior to study participation.
2. Age 18 (or age of legal consent, whichever is older) to 60 years, both inclusive.
3. Body mass index (BMI)≥18 kg/m² and ≤32 kg/m².
4. Chronic HBV infection as defined by a positive serum HBsAg for ≥6 months prior to screening.
5. Must be on NRTI therapy consisting of either entecavir, tenofovir disoproxil, tenofovir amibufenamide, or tenofovir alafenamide and with HBV-DNA<LLOQ for at least 6 months before screening (local laboratory results accepted).
6. HBsAg>100 IU/mL and ≤3,000 IU/mL at screening.
7. HBV DNA<LLOQ at screening.
8. Anti-HBS<2 IU/L at screening.
9. Serum ALT and AST≤ULN at screening.
10. Female participants must have a negative pregnancy test or confirmation of postmenopausal status.
11. Male participants with female partners of child-bearing potential must agree to meet 1 of the following contraception requirements from the time of study drug administration until 48 weeks after last study drug administration.
12. Agrees to not donate blood during the duration of the study.
13. Willing and able to comply with the study procedures and requirements, including to cease PEG-IFNα and NRTI therapy in accordance with the protocol.

Study screening laboratory tests may be repeated once for values considered to be questionable by investigator with medical monitor approval.

### Exclusion Criteria

Participants will be excluded from the study if there is evidence of any of the following at screening or prior to randomization, as appropriate:

### Medical Conditions

1. Any clinically significant chronic medical condition other than chronic HBV infection that, in the opinion of the investigator makes the participant unsuitable for participation in the study.
2. Any clinically significant acute condition such as fever (>38° C.) or acute respiratory illness within 7 days of first study drug administration.
3. Significant liver fibrosis or cirrhosis as defined by having a liver stiffness examination result of >9 kPa at screening.
4. History of clinically significant chronic liver disease from any cause other than chronic HBV infection.
5. History of hepatic decompensation, including but not limited to ascites, hepatic encephalopathy and/or esophageal or gastric varices.
6. Diagnosed or suspected hepatocellular carcinoma. Participant with screening alpha-fetoprotein (AFP) concentration ≥200 nanogram per milliliter (ng/ml) should be excluded. If the screening AFP concentration is ≥50 ng/ml and <200 ng/ml, the absence of liver mass must be documented by imaging at screening.
7. History or evidence of drug or alcohol abuse as assessed by the Investigator, within the 12 months before screening.
8. Current or past history of infection with human immunodeficiency virus (HIV), hepatitis C virus (HCV) or hepatitis Delta virus (HDV).
9. History of thyroid disease not adequately controlled on prescribed medications, or clinically significant signs of thyroid dysfunction as determined by the Investigator at screening.
10. Known history of immunological function impairment, including but not limited to:
   a) Autoimmune disease (e.g., rheumatoid arthritis, immune thrombocytopenia purpura, autoimmune hepatitis, etc); OR
   b) Primary immunodeficiency disorders (e.g., common variable immune deficiency, defective phagocytic cell function and neutropenia syndromes, and complement deficiency); OR
   c) Secondary immunodeficiency disorders (e.g., resulting from acquired immunodeficiency syndrome caused by human immunodeficiency virus (HIV) infection, solid organ transplant, and splenectomy).
11. Uncontrolled diabetes mellitus or uncontrolled hypertension as determined by the Investigator; or diagnosis of advanced stage heart failure (New York Heart Association Class III or Class IV or unstable angina).
12. Previous or current psychiatric condition that precludes compliance with the protocol. Specifically excluded are persons with severe psychiatric disorders as determined by the Investigator, including, but not limited to schizophrenia, psychosis, severe depression, bipolar illness, severe anxiety, ongoing risk for suicide, or history of suicide attempt or gesture within the past 5 years.
13. History of cancer requiring locoregional or systemic therapy within 5 years of randomization or has current disease. Participants with completely surgically resected tumors with no evidence of disease for more than 3 years, or a history of low risk basal cell carcinoma will be accepted (low risk being defined as 1) location on the trunk of the body, arms, legs, cheeks, forehead, temples, scalp, neck, or chin, 2) <2 cm in size, 3) nodular or superficial, 4) primary cancer that has not come back after treatment, 5) edge of the cancerous area is clear and smooth, and 6) not located in or around nerves).

### Prior/Concomitant Medications

14. Any HBV vaccines (licensed or experimental) within 5 years prior to screening.
15. Received any of the following treatments within 12 months prior to screening:
   a) Small interfering RNAs or antisense oligonucleotides
   b) Interferon-based therapy
16. Received any of the following treatments within 6 months before screening:
   a) Telbivudine
   b) Granulocyte colony-stimulating factor (G-CSF), granulocyte-macrophage colony-stimulating factor (GM-CSF), recombinant human interleukin-11 (oprelvekin), or erythropoietin (EPO)
17. Received blood products or immunoglobin within 90 days before study drug administration at the Day 1 visit or likely to require blood products during the study.
18. Received any of the following treatments within 30 days before study drug administration at the Day 1 visit:
   a) Myelotoxic drugs such as systemic cytostatic agents (i.e., cancer chemotherapeutic medications)
   b) Systemic immunosuppressant (including but not limited to corticosteroids at a prednisolone equivalent dose >40 mg/day)
   c) Immunization with attenuated vaccines (e.g., Mumps, Measles, Rubella)
19. Participated in another clinical trial with an investigational drug or a biologic within 30 days or within 5 drug half-lives (whichever is longer) before study drug administration at the Day 1 visit. Participants must agree not to take part in any other study at any time during their participation in this study, inclusive of the follow-up period and NRTI Discontinuation Monitoring Period.

### Discontinuation of NRTI therapy

Upon participants completing Week 88 visits, the investigator will evaluate whether participants are eligible for discontinuation of NRTI therapy based on the following NrtI discontinuation criteria:
HBsAg<LLOQ AND
HBV DNA<LLOQ AND
Undetectable HBeAg AND
ALT≤2×ULN.

Participants who meet all of the above criteria for at least two consecutive visits at Week 88 are eligible to discontinue from NrtI therapy. Investigator should confirm participants' eligibility as early as possible within 2 weeks post Week 88 visit. Participants who discontinue NrtI will enter the NrtI Discontinuation Monitoring Period of the study within 1 week of the investigator confirming eligibility. Participants will not take their NRTI medications on the first day of the NrtI Discontinuation Monitoring Period and will be followed for an additional 24 weeks.

Although for purposes of clarity of understanding the foregoing embodiments are described in some detail by way of illustration and example, it will be readily apparent to those skilled in the art that certain changes and modifications may made thereto without departing from the spirit or scope of the appended claims. Only such limitations as appear in the appended claims should be placed on any claimed invention.

All publications, patents, and patent applications cited in this specification are herein incorporated by reference as if each publication, patent, or patent application were specifically and individually indicated to be incorporated by reference.

1. A method for treating a hepatitis B viral (HBV) infection in a subject with chronic hepatitis B wherein the subject has an HBV antibody concentration below 2 IU/L, the method comprising:
a) administering to the subject an HBV surface antigen (HBsAg); and
b) administering to the subject an HBV therapy wherein it has been determined that the HBV antibody concentration in the subject is at least 2 IU/L after administration of the HBsAg.

2-20. (canceled)

21. A method for excluding subjects with chronic hepatitis B (CHB) from a clinical study, the method comprising (a) screening a group of CHB subjects by administering an HBV surface antigen (HBsAg) to the CHB subjects, wherein each CHB subject has a predetermined baseline HBV antibody concentration prior to administration of the HBsAg and wherein each CHB subject who does not respond to the HBsAg administration with a post-baseline HBV antibody concentration greater than their baseline HBV antibody concentration is excluded from the clinical study, and (b) conducting the clinical study wherein a HBV therapy is administered to the CHB subjects who are not excluded from the clinical study.

22-26. (canceled)

27. A method for treating a hepatitis B viral (HBV) infection in a responder subject with chronic hepatitis B (CHB), the method comprising:
a) determining a baseline HBV antibody concentration in a subject with CHB prior to administering an HBV surface antigen (HBsAg) to the subject;
b) administering the HBsAg to the subject;
c) determining a post-baseline HBV antibody concentration in the subject after being administered the HBsAg, and identifying the subject as a responder subject if the post-baseline HBV antibody concentration is greater than the baseline HBV antibody concentration; and
d) administering the HBV therapy to the responder subject.

28-33. (canceled)

34. A method for treating an HBV infection in a subject with chronic hepatitis B, the method comprising:
a) administering an HBV therapy to the subject wherein it has been determined that a post-baseline HBV antibody concentration in the subject after being administered an HBsAg is greater than a baseline HBV antibody concentration in the subject prior to being administered the HBsAg.

35-61. (canceled)

62. The method of claim 1, wherein an initial dose of the HBV therapy is administered after at least 4 doses of the HBsAg have been administered to the subject.

63. The method of claim 62, wherein the HBsAg is administered at 20 μg to 100 μg per dose once every two weeks (Q2W), once every three weeks (Q3W) or once every four weeks (Q4W).

64. The method of claim 62, wherein the HBsAg comprises virus-like particles (VLPs) comprising HBV surface envelope proteins Pre-S1, Pre-S2 and S.

65. The method of claim 1, wherein the initial dose of the HBV therapy is administered at least 8 weeks after the initial dose of the HBsAg.

66. The method of claim 1, wherein the HBV therapy is selected from the group consisting of an anti-HBsAg siRNA, interferon alfa (IFNα), pegylated interferon alfa (PEG-IFNα), an HBV-neutralizing mAb, and combinations thereof.

67. The method of claim 1, the HBV therapy comprises an anti-HBsAg siRNA administered to the subject at 100 mg to 400 mg per dose once every two weeks (Q2W), once every three weeks (Q3W), once every four weeks (Q4W), once every five weeks (Q5W) or once every six weeks (Q6W).

68. The method of claim 1, wherein the HBV antibody concentration is a serum anti-HBs concentration.

69. The method of claim 1, wherein the HBV therapy excludes the HBsAg.

70. The method of claim 1, wherein the HBV therapy is administered to the subject if it has been determined that the HBV antibody concentration in the subject is at least 10 IU/L after administration of the HBsAg.

71. The method of claim 21, wherein each CHB subject who does not respond to the HBsAg administration with a post-baseline HBV antibody concentration 5 times greater than their baseline HBV antibody concentration is excluded from the clinical study.

72. The method of claim 27, wherein an initial dose of the HBV therapy is administered after at least 4 doses of the HBsAg have been administered to the subject.

73. The method of claim 72, wherein the HBsAg is administered at 20 μg to 100 μg per dose once every two weeks (Q2W), once every three weeks (Q3W) or once every four weeks (Q4W).

74. The method of claim 72, wherein the HBsAg comprises virus-like particles (VLPs) comprising HBV surface envelope proteins Pre-S1, Pre-S2 and S.

75. The method of claim 27, wherein the initial dose of the HBV therapy is administered at least 8 weeks after the initial dose of the HBsAg.

76. The method of claim 27, wherein the HBV therapy is selected from the group consisting of an anti-HBsAg siRNA, interferon alfa (IFNα), pegylated interferon alfa (PEG-IFNα), an HBV.neutralizing mAb, and combinations thereof.

77. The method of claim 27, the HBV therapy comprises an anti-HBsAg siRNA administered to the responder subject at 100 mg to 400 mg per dose once every two weeks (Q2W), once every three weeks (Q3W), once every four weeks (Q4W), once every five weeks (Q5W) or once every six weeks (Q6W).

78. The method of claim 27, wherein the HBV antibody concentration is a serum anti-HBs concentration.

79. The method of claim 27, wherein the HBV therapy excludes the HBsAg.

80. The method of claim 27, wherein the subject is identified as a responder subject if the post-baseline HBV antibody concentration is 5 times greater than the baseline HBV antibody concentration.

81. The method of claim 34, wherein an initial dose of the HBV therapy is administered after at least 4 doses of the HBsAg have been administered to the subject, and wherein the HBsAg is administered at 20 μg to 100 μg per dose once every two weeks (Q2W), once every three weeks (Q3W) or once every four weeks (Q4W).

82. The method of claim 81, wherein the HBsAg comprises virus-like particles (VLPs) comprising HBV surface envelope proteins Pre-S1, Pre-S2 and S.

83. The method of claim 81, wherein the HBV therapy is selected from the group consisting of an anti-HBsAg siRNA, interferon alfa (IFNα), pegylated interferon alfa (PEG-IFNα), an HBV-neutralizing mAb, and combinations thereof.

84. The method of claim 81, wherein the HBV antibody concentration is a serum anti-HBs concentration.
